(19) United States
(12) Patent Application Publication
(10) Pub. No.: US 2007/0016242 A1
(43) Pub. Date: Jan. 18, 2007

(54) Percutaneous device with multiple expandable struts

(76) Inventor: Henry M. Israel, Baei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/180,547

(22) Filed: Jul. 14, 2005

(51) Int. Cl. A61M 29/00

(52) U.S. Cl. 606/198

(57) ABSTRACT

A percutaneous device including a body including a plurality of deformable struts, each strut having a length and a width, the length of the strut extending along an axial length of the body and the width of the strut extending about a perimeter of the body, each strut being outwardly expandable to an expanded orientation in which the length of the strut deforms into a proximal outwardly extending portion and a distal outwardly extending portion with a middle portion extending between the proximal and distal outwardly extending portions, the proximal and distal outwardly extending portions being non-parallel to an axial axis of the body and the middle portion having a surface parallel to the axial axis of the body, wherein the deformable struts are spaced from one another along the axial length and about the perimeter of the body.

### FIELD OF THE INVENTION

The present invention relates generally to devices for percutaneous transluminal coronary angioplasty (PTCA), such as guidewires and stents, and particularly to such a device with multiple expandable struts.

### BACKGROUND OF THE INVENTION

A stent is a well known device used to support an intraluminal wall, used in procedures, such as but not limited to, percutaneous transluminal coronary angioplasty (PTCA). Various types of stent architectures are known in the art, including braided stents (filaments or wires, wound or braided into a particular configuration), or mesh stents (metal mesh bent or formed into a particular shape), among others. Various types and families of expansion devices are known in the art, including hydraulic expansion, cutting and scoring balloons. These types of devices generally pass over a guide wire and are therefore larger then a normal guide wire.

In a typical stent application, the site in the body lumen at which the stent will be applied is either already expanded, or is expanded with the stent on a balloon or by the stent itself. If the body lumen is closed, neither the expansion device nor the stent can be introduced and the lumen must be opened and expanded prior to the entrance of a stent introducing system.

Typically, a stent may be restrained in a radially compressed configuration by a sheath or catheter, and delivered by an introducer to the site where it is required. The introducer may pass over a guidewire (like a monorail) that has been entered through the patient's skin, or through a blood vessel exposed by minor surgical means. When the introducer has been moved into the open body lumen to the stent deployment location, the introducer may be retained or anchored using a device similar to that described in U.S. patent application Ser. No. 11/033855 to Henry Israel. Afterwards, the introducer may be manipulated to cause the stent to be released. The stent expands to a predetermined diameter at the deployment location, and the introducer and anchor is withdrawn. Stent expansion may be effected by spring elasticity, balloon expansion, or by the self-expansion of a thermally or stress-induced return of a shape memory alloy (such as a nickel-titanium alloy, e.g., NITINOL) to a pre-conditioned expanded configuration.

In locations where the lumen is extremely narrow or occluded the standard guidewire, balloon, stent solution is too large in diameter to provide effective results. The present invention may be used as an effective solution in such a situation.

### SUMMARY OF THE INVENTION

The present invention seeks to provide a percutaneous device with multiple, outwardly expanding struts, as is described more in detail hereinbelow. In one non-limiting embodiment, the percutaneous device may be expanded in a gradually wider orientation (somewhat in the aerodynamic shape of a nose of an airplane) to help break occlusions. The percutaneous device may be an expandable guidewire, which is not normally left in a body lumen, but may also comprise a device, which is left in the body lumen, such as a stent.

There is thus provided in accordance with an embodiment of the present invention a percutaneous device including a body including a plurality of deformable struts, each strut having a length and a width, the length of the strut extending along an axial length of the body and the width of the strut extending about a perimeter of the body, each strut being outwardly expandable to an expanded orientation in which the length of the strut deforms into a proximal outwardly extending portion and a distal outwardly extending portion with a middle portion extending between the proximal and distal outwardly extending portions, the proximal and distal outwardly extending portions being non-parallel to an axial axis of the body and the middle portion having a surface parallel to the axial axis of the body, wherein the deformable struts are spaced from one another along the axial length and about the perimeter of the body.

In the expanded orientation some of the deformable struts may protrude outwards from the body more than some of the other deformable struts. For example, the most distal deformable strut may protrude outwards from the body less than the next proximal deformable strut. Further or as another example, the deformable struts may protrude gradually outwards more from the body in a proximal direction axially along the body. The ensemble of expandable, deformable, struts forms a self-anchoring assembly with an ever-expanding diameter proceeding away from the distal end of the device. The radial force of the struts is such as to open an occluded lumen in an ever-increasing manner as the device is repeatedly advanced and opened to produce a uniformly clear lumen.

In one non-limiting embodiment of the present invention the deformable struts may be constructed of a shape memory material, wherein the struts are capable of self-expansion due to at least one of thermally or stress-induced deformations of the shape memory material. Alternatively, the deformable struts may be balloon expandable. As another alternative, the body may be assembled with a tube and arranged for relative axial motion therewith, there being a stop on at least one of the body and the tube that does not permit axial motion therepast, and wherein a wire is connected to at least one of the body and the tube, wherein axial force of the wire moves at least one of the body and the tube against the stop and causes the deformable struts to buckle outwards to the expanded orientation. The device may be expanded and contracted without complete withdrawal in several successive or separate locations to provide a clear lumen(s).

In another non-limiting embodiment of the present invention the deformable struts may be combined with an inflatable balloon in such a manner that the balloon will center the device in the lumen preventing rupture of the lumen wall.

In another non-limiting embodiment of the present invention the deformable struts may be combined with a balloon, catheter or other device such as to restrain the expandable struts that are not within the occlusion, thus reducing the risk of damage to the lumen walls. In this embodiment, the shortened and gradual expansion of the expandable struts penetrating the occlusion causes the walls of the occlusion to be softened, and the lumen is opened with a gentle undulating motion from the repeated advancement cycle.

In another non-limiting embodiment of the present invention the deformable struts may be combined with a balloon, catheter or other device such as to restrain the expandable struts that are not within the occlusion. In this embodiment, the restraint in effect reduces the strut ensemble to a single active radial set, which may be extended into the occlusion and expanded.

### BRIEF DESCRIPTION OF DRAWINGS

The present invention will be further understood and appreciated from the following detailed description taken in conjunction with the drawing in which:

FIGS. 1A and 1B are simplified side-view and end-view illustrations, respectively, of a percutaneous device, constructed and operative in accordance with an embodiment of the invention, in a contracted orientation;

FIGS. 2A and 2B are simplified side-view and end-view illustrations, respectively, of the percutaneous device in an expanded orientation;

FIG. 2C is a simplified end-view illustration of the percutaneous device in an alternative expanded orientation; and

FIG. 3 is a simplified side-view illustration of the percutaneous device used in conjunction with a balloon expanding device, in accordance with an embodiment of the invention.

### DETAILED DESCRIPTION OF EMBODIMENTS

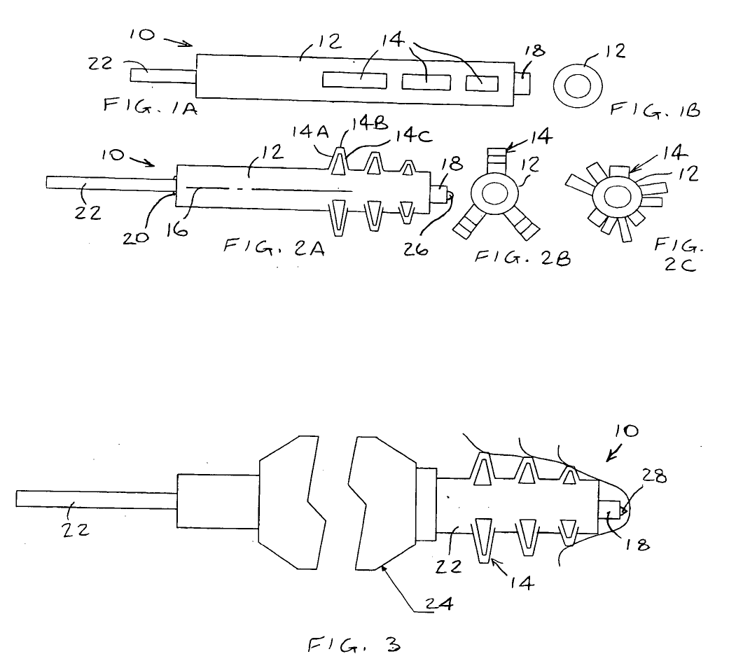

Reference is now made to FIGS. 1A-2B, which illustrates a percutaneous device 10, constructed and operative in accordance with an embodiment of the invention.

Percutaneous device 10 may include a body 12 that has a plurality of deformable struts 14. Each strut 14 has a length and a width—the length extends along an axial length of the body 12 and the width extends about a perimeter of the body 12. FIGS. 1A and 1B illustrate percutaneous device 10 in a contracted orientation.

As seen in FIGS. 2A and 2B, each strut 14 is outwardly expandable to an expanded orientation in which the length of the strut 14 deforms into a proximal outwardly extending portion 14A and a distal outwardly extending portion 14C with a middle portion 14B extending between the proximal and distal outwardly extending portions 14A and 14C. The proximal and distal outwardly extending portions 14A and 14C may be non-parallel to an axial axis 16 of the body 12 and the middle portion 14B may have a surface parallel to the axial axis 16. This surface of the middle portion 14B may be long or short (even a point), curved or straight. The deformable struts 14 may be spaced from one another along the axial length and about the perimeter of the body 12. As seen by comparing FIG. 2B and FIG. 2C, the struts 14 may be spaced in different patterns about the perimeter of the body 12.

In the expanded orientation some of the deformable struts 14 may protrude outwards from the body more than some of the other deformable struts 14. For example, the most distal deformable strut 14 may protrude outwards from the body 12 less than the next proximal deformable strut. Further or as another example, the deformable struts 14 may protrude gradually outwards more from the body 12 in a proximal direction axially along the body 12. In this manner, the percutaneous device 10 may be expanded in a gradually wider orientation (somewhat in the aerodynamic shape of a nose of an airplane) to help break occlusions and open the lumen.

In one non-limiting embodiment of the present invention the deformable struts 14 may be constructed of a shape memory material, wherein the struts 14 are capable of self-expansion due to at least one of thermally or stress-induced deformations of the shape memory material. Alternatively, the deformable struts 14 may be balloon expandable. Accordingly, percutaneous device 10 may be made of a durable material, such as but not limited to, a plastic, a shape memory alloy (such as a nickel-titanium alloy, e.g., NITINOL), or stainless steel (e.g., stainless steel 316L), which may be coated with a material such as polytetrafluoroethylene (PTFE). Percutaneous device 10 may be introduced with any sheathing device, such as but not limited to, a catheter, balloon or guiding catheter delivery catheter over a guidewire, as is well known in the art.

As another alternative construction, the body 12 may be assembled with one or more tubes 18 (one is shown in the illustrated embodiment) and arranged for relative axial motion therewith. A stop 20 may be on body 12 or on tube 18 that does not permit axial motion therepast. One or more wires 22 (one is shown in the illustrated embodiment) may be connected to body 12 or tube 18. The wire 22 may be a pull wire. Alternatively, wire 22 may be any relatively slender, relatively stiff element of arbitrary cross section, which can push an object and substantially not buckle. In any case, the axial force of wire 22 moves the body. 12 or tube 18 against stop 20 and causes the deformable struts 14 to buckle outwards to the expanded orientation. The buckling may be gradual, that is, the most distal struts may buckle outwards first, followed by the next most proximal struts, followed by the next most proximal struts and so on.

Reference is now made to FIG. 3, which illustrates the percutaneous device 10 assembled with a balloon expanding device 24, in accordance with an embodiment of the invention. Balloon expanding device 24 may be, without limitation, a wire mesh or braided stent, for example. Stent 10 may be balloon-expandable, constructed from a suitable material, such as but not limited to, stainless steel 316L.

The percutaneous device 10 may also include an occlusion breaking element at a distal end thereof (e.g., on tube 18 or on the body 12), which may help break up (grind, chop, slice, pulverize or otherwise break up) occlusions in the body lumen. For example, as shown in FIG. 2A, the occlusion breaking element may comprise a ball 26. Another example is shown in FIG. 3, where the occlusion-breaking element may comprise a sharp pointed element 28.

One method of using the percutaneous device 10 includes introducing the percutaneous device 10 into a body lumen (e.g., blood vessel, bile duct, urinary tract, etc.) until the distal end of the device meets an occlusion. The strut or struts at the distal end may be expanded to open the occlusion and the lumen. The percutaneous device may then be advanced further into the lumen and once again the next set of struts may be expanded to open the occlusion as the device is moved forward. Thus the distal end of the device is self anchoring at the occlusion as the proximal end of the device opens the occlusion. Additionally or alternatively, the balloon-expanding device 24 may be used to center, restrain and anchor the percutaneous device 10 as the struts or the balloon open the occlusion, thereby preventing rupture of the lumen wall.

Accordingly, in one method of the invention, the percutaneous device 10 may be expanded and contracted without complete withdrawal in several successive or separate locations to provide a clear lumen(s).

In another non-limiting embodiment of the present invention, the balloon expanding device 24 (or catheter or other similar device) may be used to restrain expansion of the expandable struts 14 that are not within the occlusion, thus reducing the risk of damage to the lumen walls. In this embodiment, the shortened and gradual expansion of the expandable struts 14 penetrating the occlusion causes the walls of the occlusion to be softened, and lumen is opened with a gentle undulating motion from the repeated advancement cycle. The restraining device in effect reduces the strut ensemble to a single active radial set, which may be extended into the occlusion and expanded.

It is appreciated that various features of the invention which are, for clarity, described in the contexts of separate embodiments, may also be provided in combination in a single embodiment. Conversely, various features of the invention, which are, for brevity, described in the context of a single embodiment, may also be provided separately or in any suitable subcombination.

What is claimed is:

1. A percutaneous device comprising:
a body comprising a plurality of deformable struts, each strut having a length and a width, the length of the strut extending along an axial length of the body and the width of the strut extending about a perimeter of the body, each strut being outwardly expandable to an expanded orientation in which the length of the strut deforms into a proximal outwardly extending portion and a distal outwardly extending portion with a middle portion extending between the proximal and distal outwardly extending portions, the proximal and distal outwardly extending portions being non-parallel to an axial axis of the body and the middle portion having a surface parallel to the axial axis of the body, wherein the deformable struts are spaced from one another along the axial length and about the perimeter of the body.

2. The percutaneous device according to claim 1, wherein in the expanded orientation some of said deformable struts protrude outwards from the body more than some of the other deformable struts.

3. The percutaneous device according to claim 2, wherein in the expanded orientation the most distal deformable strut protrudes outwards from the body less than the next proximal deformable strut.

4. The percutaneous device according to claim 2, wherein in the expanded orientation the deformable struts protrude gradually outwards more from the body in a proximal direction axially along the body.

5. The percutaneous device according to claim 1, wherein said deformable struts are constructed of a shape memory material, wherein the struts are capable of self-expansion due to at least one of thermally or stress-induced deformations of the shape memory material.

6. The percutaneous device according to claim 1, wherein said deformable struts are balloon expandable.

7. The percutaneous device according to claim 1, wherein said body is assembled with at least one tube and arranged for relative axial motion therewith, there being a stop on at least one of said body and said at least one tube that does not permit axial motion therepast, and wherein at least one wire is connected to at least one of said body and said at least one tube, wherein axial force of said at least one wire moves at least one of said body and said at least one tube against said stop and causes said deformable struts to buckle outwards to the expanded orientation.

8. The percutaneous device according to claim 1, wherein said body comprises a guidewire.

9. The percutaneous device according to claim 8, wherein said guide wire is assembled with a balloon expanding device.

10. The percutaneous device according to claim 9, wherein said balloon expanding device is adapted to anchor the percutaneous device during opening of an occlusion.

11. The percutaneous device according to claim 9, wherein said balloon expanding device is adapted to center the percutaneous device during opening of an occlusion.

12. The percutaneous device according to claim 1, wherein an occlusion breaking element is disposed on a distal end of said percutaneous device, adapted to help break up occlusions in a body lumen.

13. The percutaneous device according to claim 1, further comprising a device adapted to restrain expansion of a portion of the expandable struts.

14. A method for deploying a percutaneous device comprising:
introducing a percutaneous device comprising expandable struts into a body lumen until a distal end of the percutaneous device meets an occlusion;
expanding the struts outwards at the distal end to open the occlusion and the body lumen; and
advancing the percutaneous device further into the body lumen and expanding another set of struts outwards to open another occlusion.

15. The method according to claim 14, wherein the distal end of the percutaneous device is anchored at the occlusion as the proximal end of the device opens the occlusion.

16. The method according to claim 14, wherein a balloon expanding device is used to anchor the percutaneous device while opening the occlusion.

17. The method according to claim 14, wherein a balloon expanding device is used to center the percutaneous device while opening the occlusion.

18. The method according to claim 14, further comprising restraining expansion of the expandable struts that are not within the occlusion.

19. The method according to claim 14, comprising gradually expanding the expandable struts to penetrate and soften the occlusion, and repeatedly expanding the expandable struts with a gentle undulating motion to open the body lumen.
